(19) United States
(12) Patent Application Publication
(10) Pub. No.: US 2017/0273548 A1
(43) Pub. Date: Sep. 28, 2017

(54) LASER SCANNING OBSERVATION APPARATUS

(71) Applicant: OLYMPUS CORPORATION, Tokyo (JP)

(72) Inventor: Yuhei TAKATA, Tokyo (JP)

(73) Assignee: OLYMPUS CORPORATION, Tokyo (JP)

(21) Appl. No.: 15/621,407

(22) Filed: Jun. 13, 2017

Related U.S. Application Data
(63) Continuation of application No. PCT/JP2014/006264, filed on Dec. 16, 2014.

(51) Int. Cl. A61B 1/00

(52) U.S. Cl. CPC A61B 1/00172; A61B 1/00009; A61B 1/00186

(57) ABSTRACT

Color information with higher sensitivity than color information in a primary color system is detected. A laser scanning observation apparatus includes at least three light sources, a multiplexer, and a controller. The at least three light sources emit pulsed laser light with different wavelengths within the visible light spectrum. The multiplexer combines the laser light emitted from the at least three light sources. The controller causes different combinations of two light sources among the at least three light sources to emit laser light in sequence and periodically.

### CROSS-REFERENCE TO RELATED APPLICATION

The present application is a Continuing Application based on International Application PCT/JP2014/006264 filed on Dec. 16, 2014, the entire disclosure of which is incorporated herein by reference.

### TECHNICAL FIELD

This disclosure relates to a laser scanning observation apparatus capable of detecting light of a variety of wavelengths.

### BACKGROUND

A known optical scanning observation apparatus is capable of scanning an object of observation and capturing an image by oscillating an optical fiber that emits light (see JP 2010-142605 A (PTL 1)).

### CITATION LIST

### Patent Literature

PTL 1: JP 2010-142605 A

### SUMMARY

In the optical scanning observation apparatus disclosed in PTL 1, an object of observation is irradiated by light, and scattered light from the object of observation is separated into components. An image is then formed using the color information component detected for each of the colors R, G, and B. There has been a desire, however, for detecting color information with a higher sensitivity than the color information in a primary color system such as RGB.

### BRIEF DESCRIPTION OF THE DRAWINGS

In the accompanying drawings:

FIG. 1 is a functional block diagram schematically illustrating the internal configuration of a laser scanning observation apparatus that includes an optical scanning unit according to one of the embodiments of this disclosure;

FIG. 2 is a functional block diagram schematically illustrating the internal configuration of the light source unit in FIG. 1;

FIG. 3 is an external view schematically illustrating the optical scanning endoscope apparatus body in FIG 1;

FIG. 4 is a cross-sectional diagram illustrating an enlargement of the tip of the optical scanning endoscope apparatus body in FIG. 1;

FIG. 5 is an enlarged perspective view around the driver in FIG. 4;

FIG. 6 is a functional block diagram schematically illustrating the internal configuration of the detector in FIG. 1;

FIG. 7 is a timing chart illustrating driving of the light source unit by the controller in a first illumination mode; and

FIG. 8 is a timing chart illustrating driving of the light source unit by the controller in a second illumination mode.

### DETAILED DESCRIPTION

Embodiments are described below with reference to the drawings.

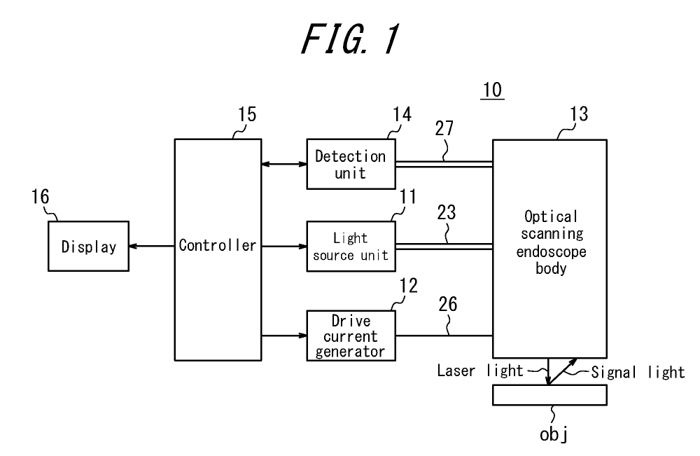

FIG. 1 is a functional block diagram schematically illustrating the internal configuration of a laser scanning observation apparatus according to one of the embodiments of this disclosure.

A laser scanning observation apparatus 10 for example is a laser scanning endoscope apparatus and is configured to include a light source unit 11, a drive current generator 12, an optical scanning endoscope body 13, a detection unit 14, a controller 15, and a display 16.

As described below, the light source unit 11 emits laser light and supplies the laser light to the optical scanning endoscope body 13. The drive current generator 12 transmits a drive signal necessary for scanning an object obj to the optical scanning endoscope body 13. The optical scanning endoscope body 13 scans the object obj with the laser light and propagates the signal light obtained by the scan to the detection unit 14. The detection unit 14 converts the propagated signal light to an electrical signal. The controller 15 synchronously controls the light source unit 11, the drive current generator 12, and the detection unit 14, processes the electrical signal output by the detection unit 14, synthesizes an image, and displays the image on the display 16.

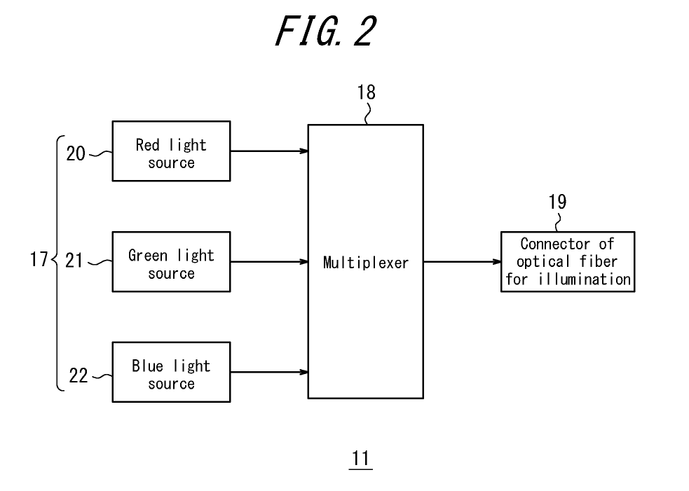

As illustrated in FIG. 2, the light source unit 11 is configured to include at least three light sources 17, a multiplexer 18, and a connector 19 of an optical fiber for illumination.

The at least three light sources 17 emit pulsed laser light with different wavelengths within the visible light spectrum. In this embodiment, the at least three light sources 17 are constituted by three light sources: a red light source 20, a green light source 21, and a blue light source 22. The red light source 20 may, for example, be a red laser that emits red laser light with a wavelength of 640 nm. The green light source 21 may, for example, be a green laser that emits green laser light with a wavelength of 532 nm. The blue light source 22 may, for example, be a blue laser that emits blue laser light with a wavelength of 445 nm.

The multiplexer 18 may, for example, be configured by a dichroic mirror and a fiber combiner and combines the red laser light, green laser light, and blue laser light respectively emitted by the red light source 20, green light source 21, and blue light source 22.

An optical fiber for illumination is provided in the optical scanning endoscope body 13. The connector 19 of the optical fiber for illumination optically connects to the optical fiber for illumination and supplies the laser light output from the multiplexer 18 to the optical fiber for illumination.

On the basis of control by the controller 15, the drive current generator 12 (see FIG. 1) generates a drive signal that displaces the emission end of an optical fiber 23 for illumination in a spiral. The drive current generator 12 supplies the drive signal to a driver provided in the optical scanning endoscope body 13.

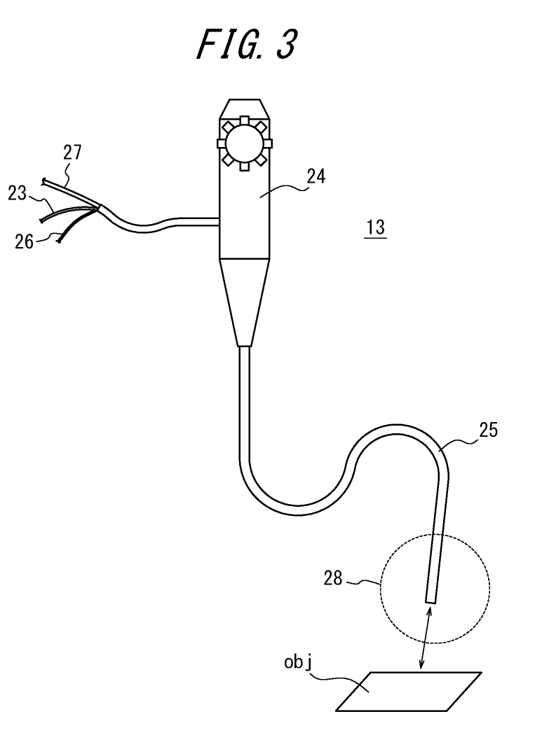

As illustrated in FIG. 3, the optical scanning endoscope body 13 includes an operation part 24 and an insertion part 25. One end of the operation part 24 is integrally connected to the base end of the insertion part 25.

The optical scanning endoscope body 13 includes the optical fiber 23 for illumination, a wiring cable 26, and an optical fiber bundle 27 for detection. The optical fiber 23 for illumination, wiring cable 26, and optical fiber bundle 27 for detection pass from the operation part 24 through the insertion part 25 and are drawn to a tip 28 (the portion enclosed by dashes in FIG. 3) of the insertion part 25. The optical fiber 23 for illumination is connected to the connector 19 of the optical fiber for illumination in the light source unit 11 at the operation part 24 side and propagates laser light to the tip 28. The wiring cable 26 is connected to the drive current generator 12 at the operation part 24 side and transmits drive signals to the driver provided at the tip 28, The optical fiber bundle 27 for detection is connected to the detection unit 14 at the operation part 24 side and propagates the signal light obtained at the tip 28 to the detection unit 14.

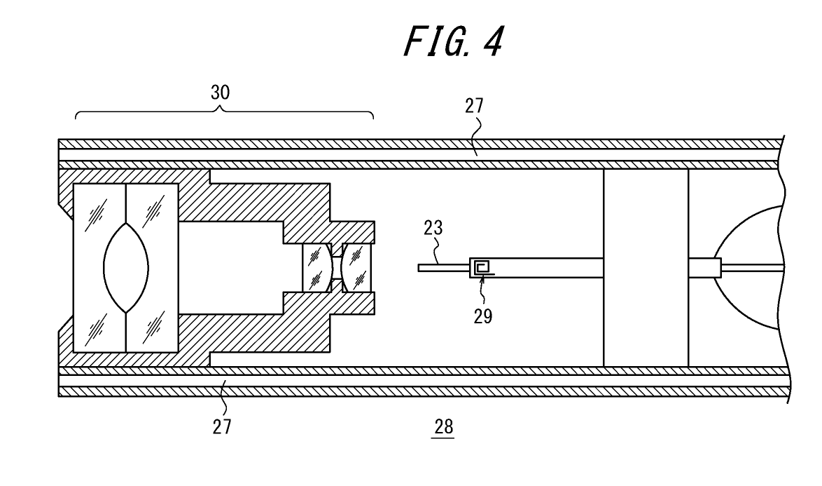

FIG. 4 is a cross-sectional diagram illustrating an enlargement of the tip 28 of the optical scanning endoscope body 13 in FIG 1. A driver 29, an illumination optical system 30, and a non-illustrated detection lens are provided at the tip 28. Also, the optical fiber 23 for illumination and the optical fiber bundle 27 for detection extend to the tip 28.

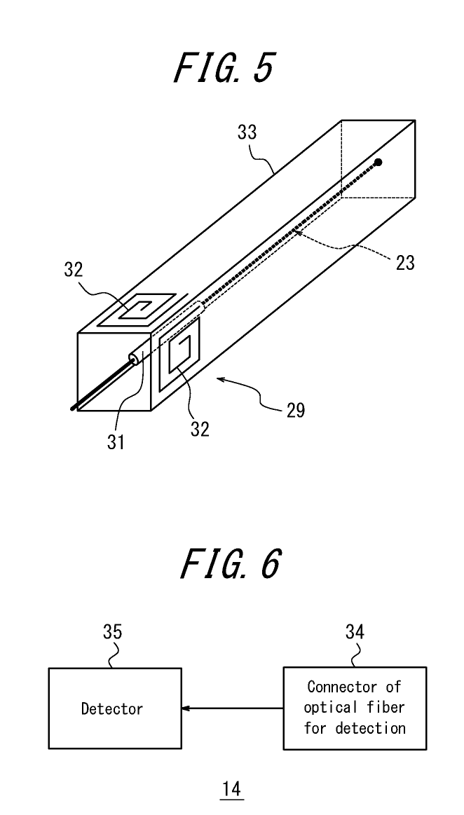

The driver 29 may, for example, be an electromagnetic actuator configured by a permanent magnet 31 (see FIG. 5) and coils 32 for generating a deflection magnetic field. The permanent magnet 31 is tubular and is attached to the optical fiber 23 for illumination with the optical fiber 23 for illumination inserted therethrough. The optical fiber 23 for illumination is supported by a square tube 33 in a state allowing oscillation of the area near the emission end, including the permanent magnet 31. The coils 32 for generating a deflecting magnetic field are provided on each of the four sides of the square tube 33. As a result of the drive signal supplied by the drive current generator 12, the coils 32 for generating a deflecting magnetic field generate a magnetic field and deflect the permanent magnet 31 along with the emission end of the optical fiber 23 for illumination in two directions. The driver 29 drives the emission end of the optical fiber 23 for illumination on the basis of the drive signal while increasing the amplitude from zero to the maximum amplitude and then reducing the amplitude back to zero during one frame, thereby deflecting the emission end. By vibrating the emission end of the optical fiber 23 for illumination as described above in two different directions, the driver 29 scans the object obj in a spiral with laser light emitted from the emission end.

The illumination optical system 30 (see FIG. 4) is disposed at the extreme end of the tip 28 of the insertion part 25, i.e. in the direction of emission from the emission end of the optical fiber 23 for illumination. The illumination optical system 30 is configured so that laser light emitted from the emission end of the optical fiber 23 for illumination is roughly concentrated on the object obj.

Upon laser light being concentrated on the object obj light may interact with and be reflected, scattered, or refracted by the object obj, or fluorescent light may be generated. The detection lens is disposed so as to capture the light that interacted with the object obj, the fluorescent light, and the like as signal light and to concentrate and combine the signal light on the optical fiber bundle 27 for detection disposed behind the detection lens.

As illustrated in FIG. 6, the detection unit 14 is configured to include a connector 34 of the optical fiber for detection and a detector 35. The connector 34 of the optical fiber for detection optically connects to the optical fiber bundle 27 for detection and acquires signal light from the optical fiber bundle 27 for detection. The detector 35 may, for example, be an photomultiplier tube or a photodiode and detects the light intensity of the signal light.

The controller 15 (see FIG. 1) controls each part of the laser scanning observation apparatus 10. For example, as described above, the controller 15 synchronously controls the light source unit 11, the drive current generator 12, and the detection unit 14, processes the electrical signal output by the detection unit 14, and synthesizes an image.

The controller 15 drives the light source unit 11 in a first illumination mode or a second illumination mode. The first illumination mode and the second illumination mode are operation modes provided in the laser scanning observation apparatus 10. In the first illumination mode, signal light representing color information in a complementary color system is detected. In the second illumination mode, signal light representing color information in a primary color system is detected. Details are provided below on how the controller 15 drives the light source unit 11 in the first illumination mode and the second illumination mode.

The controller 15 can switch between the first illumination mode and the second illumination mode each frame. For example, the laser scanning observation apparatus 10 can be used to input a selection to prioritize either sensitivity or color reproduction for a captured image. Upon detecting input to prioritize sensitivity, the controller 15 generates an image in the first illumination mode. On the other hand, upon detecting input to prioritize color reproduction, the controller 15 generates an image in the second illumination mode.

The controller 15 can also switch between the first illumination mode and the second illumination mode within a frame. For example, the laser scanning observation apparatus 10 can prioritize sensitivity when imaging a central region centering on the optical axis of the detection lens and can prioritize color reproduction when imaging a region surrounding the central region. During such imaging, the controller 15 drives the light source unit 11 in the first illumination mode when scanning the central region and drives the light source unit 11 in the second illumination mode when scanning the surrounding region. The laser scanning observation apparatus 10 can also prioritize sensitivity when imaging the surrounding region and prioritize color reproduction when imaging the central region. During such imaging, the controller 15 drives the light source unit 11 in the second illumination mode when scanning the central region and drives the light source unit 11 in the first illumination mode when scanning the surrounding region.

A complementary color system image is based on signal light representing color information in a complementary color system, and a primary color system image is based on signal light representing color information in a primary color system. The controller 15 can convert in one or both directions between a complementary color system image and a primary color system image. For conversion between a complementary color system and a primary color system, R, G, B, Cy, Mg, and Y are taken as signal values of red light, green light, blue light, cyan light, magenta light, and yellow light, and the equations R=1−Cy, G=1−Mg, and B=1−Y are used.

When causing laser light to be emitted from two light sources in the first illumination mode, the controller 15 can adjust the ratio of laser light intensity. For example, in order to emit magenta laser light with little redness, the controller 15 controls the red light source 20 and the green light source 21 to reduce the intensity of red laser light to be below the intensity of green laser light.

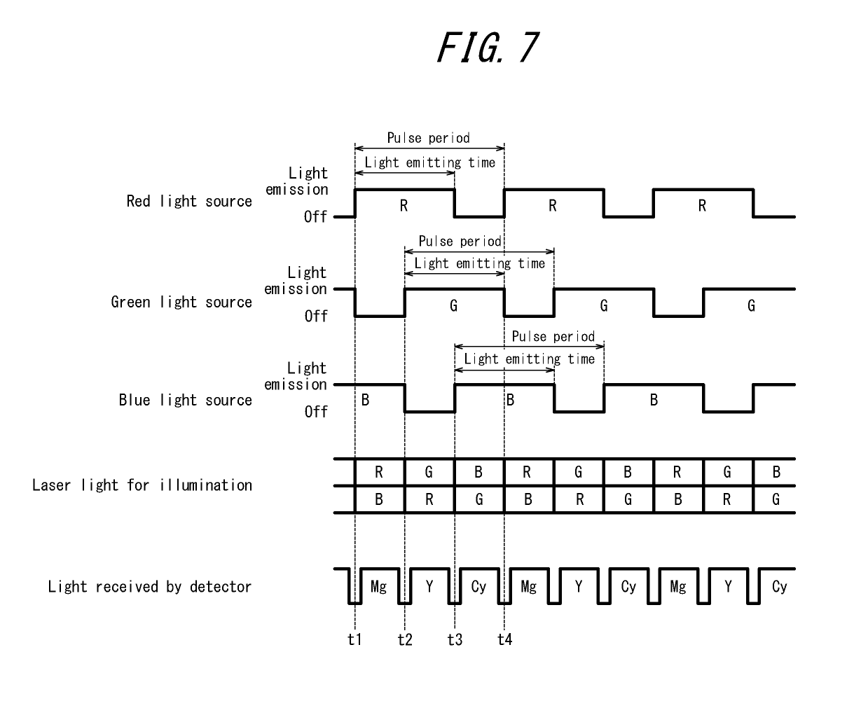

Next, driving of the light source unit 11 by the controller 15 in the first illumination mode is described. In the first illumination mode, as illustrated in FIG. 7, the controller 15 causes different combinations of two light sources among the red light source 20, green light source 21, and blue light source 22 to emit laser light in sequence and periodically. In greater detail, the controller 15 causes the red light source 20, green light source 21, and blue light source 22 to emit light with an identical pulse period and shifts the light emission start time so that two light sources have a partially overlapping light emitting time. Furthermore, the controller 15 causes the red light source 20, green light source 21, and blue light source 22 to emit light so that the interval of each light emitting time is the same.

For example, from timing t1 at which the red light source 20 starts to emit light until timing t2, at which point half of the light emitting time has elapsed, the controller 15 controls the red light source 20, green light source 21, and blue light source 22 so that the green light source 21 is off and the blue light source 22 emits light. Accordingly, during the time from timing t1 to timing t2, the light emitting times of the red light source 20 and the blue light source 22 partially overlap.

From timing t2 until timing t3, at which point half of the light emitting time has elapsed, the controller 15 controls the red light source 20, green light source 21, and blue light source 22 so that the red light source 20 emits light, the green light source 21 emits light, and the blue light source 22 is off. Accordingly, during the time from timing t2 to timing t3, the light emitting times of the red light source 20 and the green light source 21 partially overlap.

From timing t3 until timing t4, at which point half of the light emitting time has elapsed, the controller 15 controls the red light source 20, green light source 21, and blue light source 22 so that the red light source 20 is off, the green light source 21 emits light, and the blue light source 22 emits light. Accordingly, during the time from timing t3 to timing t4, the light emitting times of the green light source 21 and the blue light source 22 partially overlap.

In this way, during the time from timing t1 to timing t2, red laser light and blue laser light are combined (see the column “laser light for illumination”), and magenta laser light is output from the multiplexer 18 (see the column “light received by detector”). During the time from timing t2 to timing t3, red laser light and green laser light are combined (see the column “laser light for illumination”), and yellow laser light is output from the multiplexer 18 (see the column “light received by detector”). During the time from timing t3 to timing t4, green laser light and blue laser light are combined (see the column “laser light for illumination”), and cyan laser light is output from the multiplexer 18 (see the column “light received by detector”). From the timing t4 onward, the same control as from timing t1 to timing t4 is cyclically repeated, and magenta laser light, yellow laser light, and cyan laser light are output from the multiplexer 18 in sequence and periodically.

The controller 15 causes the detection unit 14 to detect the intensity of signal light within the respective output times of magenta laser light, yellow laser light, and cyan laser light (see the column “light received by detector”).

Furthermore, the controller 15 synthesizes an image on the basis of the signal values detected by the detection unit 14. The signal values are in the complementary color system of magenta, yellow, and cyan.

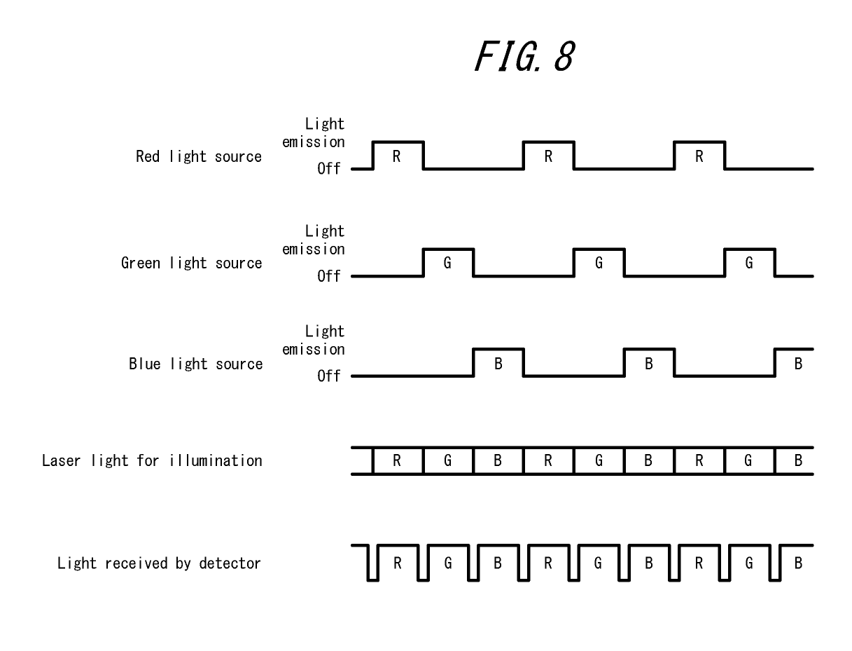

Next, driving of the light source unit 11 by the controller 15 in the second illumination mode is described. In the second illumination mode, as illustrated in FIG. 8, the controller 15 causes the red light source 20, green light source 21, and blue light source 22 to emit red laser light, green laser light, and blue laser light in sequence and periodically. Accordingly, in the second illumination mode, red laser light, green laser light, and blue laser light are output from the multiplexer 18 in sequence and periodically. The controller 15 causes the detection unit 14 to detect the intensity of signal light within the respective output times of red laser light, green laser light, and blue laser light. Furthermore, the controller 15 synthesizes an image on the basis of the color information in the primary color system for the red light, green light, and blue light detected by the detection unit 14.

The laser scanning observation apparatus of this embodiment with the above-described configuration causes different combinations of two light sources among at least three light sources in the light source unit 11 to emit laser light in sequence and periodically. Therefore, color information in a complementary color system, which has higher sensitivity than color information in a primary color system, can be detected. The laser scanning observation apparatus of this embodiment can also sequentially change the irradiated light during scanning of the object obj by emitting laser light in sequence and periodically in the different combinations of two light sources. Hence, the laser scanning observation apparatus can detect color information in a complementary color system without using a spectroscopic optical system.

The laser scanning observation apparatus of this embodiment causes all of the three light sources to emit light or to turn off with an identical pulse period so that two-thirds of the time of the pulse period becomes the light emitting time, half or less of the light emitting time of each light source overlaps the light emitting time of another light source, and a different half or less of the light emitting time of each light source overlaps the light emitting time of yet another light source. Hence, the laser scanning observation apparatus can efficiently detect color information in a sensitive complementary color system and can acquire a seamless image.

The laser scanning observation apparatus of this embodiment adjusts the ratio of intensity of the laser light emitted from two light sources that are combined. Hence, the laser scanning observation apparatus can detect color information in a complementary color system that has a desired color tone in accordance with the object of observation and the usage environment. For example, white balance adjustment can be made on the light source unit 11 side for the spectral sensitivity characteristic of the detector 35 relative to wavelength. As compared to adjusting the white balance by adjusting the gain of the signal detected by the detector 35 for each color, this approach can suppress saturation of the signal value and can suppress a reduction in the S/N ratio.

The laser scanning observation apparatus of this embodiment can also drive the light source unit 11 separately in the first illumination mode and the second illumination mode. Hence, with a simple configuration, the laser scanning observation apparatus can acquire color information in both a complementary color system and a primary color system.

The laser scanning observation apparatus of this embodiment can switch between the first illumination mode and the second illumination mode within a frame. Hence, the laser scanning observation apparatus can acquire color information with high visibility overall in accordance with the circumstances of the insertion part 25. For example, when the insertion part 25 is being inserted towards the object of observation, the subject in the central region is farther away than the tip 28. Since the intensity of acquired signal light is relatively low, sensitive imaging is required in the central region. Also, the subject in the surrounding region is closer to the tip 28 than the central region is. Since the intensity of acquired signal light is relatively high, it is considered appropriate to prioritize color reproduction in the surrounding region more than in the central region. Therefore, while the insertion part 25 is being inserted, an image with the required visibility can be captured by imaging the central region in the first illumination mode and the surrounding region in the second illumination mode. When the insertion part 25 is opposite the object of observation, the subject in the central region is near the tip 28 and can be illuminated with a sufficient amount of light. Hence, imaging that prioritizes color reproduction is required in the central region. Also, since less of the laser light for illumination reaches the surrounding region than the central region, sensitive imaging is required in the surrounding region. Therefore, when the insertion part 25 is opposite the object of observation, an image with the required visibility can be captured by imaging the central region in the second illumination mode and the surrounding region in the first illumination mode.

Although this disclosure has been described on the basis of embodiments and the accompanying drawings, it is to be noted that various changes and modifications will be apparent to those skilled in the art on the basis of this disclosure. Therefore, such changes and modifications are to be understood as included within the scope of this disclosure.

1. A laser scanning observation apparatus comprising:
at least three light sources configured to emit pulsed laser light with different wavelengths within a visible light spectrum;
a multiplexer configured to combine the laser light emitted from the at least three light sources; and
a controller configured to cause different combinations of two light sources among the at least three light sources to emit the laser light in sequence and periodically,

2. The laser scanning observation apparatus of claim 1, wherein the controller causes the at least three light sources to emit the laser light with an identical pulse period and shifts a light emission start time of each light source so that the two light sources differently combined have a partially overlapping light emitting time.

3. The laser scanning observation apparatus of claim 2, wherein
the at least three light sources comprise three light sources; and
the controller drives the three light sources so that two-thirds of a time of the pulse period becomes the light emitting time, half or less of the light emitting time of each light source overlaps the light emitting time of another light source, and a different half or less of the light emitting time of each light source overlaps the light emitting time of yet another light source.

4. The laser scanning observation apparatus of claim 1, wherein the controller adjusts a ratio of intensity of the laser light emitted from the two light sources differently combined.

5. The laser scanning observation apparatus of claim 1, wherein the controller controls the light sources in a first illumination mode and a second illumination mode, the first illumination mode causing the two light sources differently combined to emit the laser light in sequence and periodically and the second illumination mode causing each light source to emit the laser light in sequence and periodically.
